(19) United States
(12) Patent Application Publication
(10) Pub. No.: US 2023/0307220 A1
(43) Pub. Date: Sep. 28, 2023

(54) Method for Extracting cIEF-MS Profiles from m/z versus Time Arrays

(71) Applicant: DH TECHNOLOGIES DEVELOPMENT PTE. LTD., Singapore (SG)

(72) Inventor: John Warwick Silzel, Yorba Linda, CA (US)

(21) Appl. No.: 18/041,192

(22) PCT Filed: Aug. 9, 2021
(86) PCT No.: PCT/IB2021/057333

§ 371 Date: Feb. 9, 2023

Related U.S. Application Data
(60) Provisional application No. 63/064,558, filed on Aug. 12, 2020.

(51) Int. Cl. H01J 49/00; G16B 40/10

(52) U.S. Cl. CPC H01J 49/0036; G16B 40/10; H01J 49/004

(57) ABSTRACT

A 3-D array of intensity measurements measured as a function of m/z and time is received from a separation device coupled mass spectrometer. The 3-D array is converted to an intensity matrix D. The rows correspond to measured m/z or m/z related values. The columns correspond to time or time-related values. NMF is applied to the D matrix to solve for matrix M and matrix A of the equation D=MA. The NMF is applied to produce in a row (i) of the A matrix time or time-related intensity values for a peak (i) that separates the peak (i) from the 3-D array. The NMF is also applied to produce in column (i) of the M matrix m/z or m/z related intensity values corresponding to the peak (i). A profile of peak (i) is displayed from row (i) or a sum of two or more rows.

### RELATED APPLICATIONS

This application claims the benefit of U.S. Provisional Patent Application Ser. No. 63/064,558, filed on Aug. 12, 2020, the content of which is incorporated by reference herein in its entirety.

### INTRODUCTION

The teachings herein relate to separating intensity peaks from data collected in a separation device coupled mass spectrometry experiment. More particularly, the teachings herein relate to systems and methods for separating a peak or peak profile from a three-dimensional (3-D) array of intensity measurements measured as a function of m/z and time using nonnegative matrix factorization (NMF). The systems and methods disclosed herein can be performed in conjunction with a processor, controller, microcontroller, or computer system, such as the computer system of FIG. 1.

cIEF-MS Data Analysis Background

Capillary isoelectric focusing (cIEF) followed by mass spectrometry (cIEF-MS) is a hyphenated technique used to first separate a mixture of monoclonal antibodies or other proteins on the basis of their isoelectric points (pI) into isoforms. Once separated the isoforms are transferred serially to a mass spectrometer where their mass-to-charge (m/z) ratios are measured. A sample is generally a mixture of substances that yields multiple separated isoforms during cIEF, and each isoform subsequently generates multiple m/z signals because of the varying charge states. The data from a single cIEF-MS measurement is a large two-dimensional (2-D) digital array of intensity values, arranged by m/z value along one axis, and by time on the other axis. In other words, including intensity, a single cIEF-MS measurement produces three-dimensional (3-D) data, where the three dimensions are m/z, time, and intensity. A common cIEF-MS sample type is, for example, a monoclonal antibody having a parent molecular mass between 140 and 150 kilodaltons (kD).

FIG. 2 is an exemplary 3-D plot 200 from a single cIEF-MS measurement showing measured intensities as a function of measured m/z values and measured time values, upon which embodiments of the present teachings may be implemented. Specifically, in plot 200, measured intensities (counts) 210 are plotted as a function of measured time values 220 and measured m/z values 230.

One problem in cIEF-MS data analysis is that this large amount of 3-D data needs to be reduced to a 2-D plot of intensity versus time or pI value, displaying the number of isoforms discovered by the cIEF step. A 2-D plot of intensity versus time or pI value can also, for example, be referred to as a one-dimensional profile. Another problem is that a parent mass value needs to be assigned to each important signal, or peak, in the 2-D plot. Both axes of the 3-D data present that data in a form less meaningful to most scientists interested in the isoelectric points and masses of isoforms: the m/z axis presents a redundant description of the desired parent mass information, and the time axis affords no information about pI. The analysis therefore requires (a) reconstruction of the parent mass from the m/z axis, and (b) calibration of the time axis in terms of pI.

Both (a) and (b) are achieved by conventional methods by, for example, processing the 2-D array of intensities row-wise or column-wise. Both (a) and (b) are achieved, if at every point along the time axis there is only a single protein isoform present at a unique p1 value, or if every column of m/z data uniformly represents the ionic forms of a single unique parent mass. However, the separation of isoforms by cIEF is not perfect because any intensity value measured has the potential to originate from two or more isoforms having the same parent mass, two or more isoforms having unique parent masses, or two parent masses having pI values so similar that they are not well resolved. In these cases, conventional algorithms must favor either mass or pI in attempting to extract a 2-D plot from the data. Either of these approaches is biased and may miss components of interest to a scientist.

As a result, systems and methods are needed to automatically separate isoforms from the 3-D data even if there are intensity values that originate from two or more isoforms having the same parent mass, two or more isoforms having unique parent masses, or two parent masses having pI values so similar that they are not well resolved.

CE and cIEF Device Background

FIG. 3 is an exemplary schematic diagram 300 of a capillary electrophoresis (CE) system, upon which embodiments of the present teachings may be implemented. CE system 300 includes CE device 310 and detector 320. CE device 310 includes fused-silica capillary 311 with optical viewing window 312, controllable high voltage power supply 313, two electrode assemblies 314, and two buffer reservoirs 315 and 316. The ends of capillary 311 are placed in buffer reservoirs 315 and 316 and optical viewing window 312 is aligned with detector 320, when detector 320 is an optical detector. After filling capillary 311 with buffer, the sample can be injected into capillary 311.

Electrophoresis is fundamentally the movement of charged particles within an applied electric field. In CE, a sample is injected at one end of capillary 311. Detector 320 is positioned or attached to capillary 311 at the other end of capillary 311 distant from the sample. A voltage, provided by high voltage power supply 313 and two electrode assemblies 314, is applied along the length of the capillary 311.

With the electric potential applied, two separate flow effects occur. The first of these flow effects is a gross sample flow effect. The sample moves as a mass into the capillary. The second of these flow effects is the electrophoretic flow. This causes the constituents of the sample having differing electric charges to move relative to the main stream of fluid within capillary 311. The portions of the sample having differing electric charges are thereby separated in capillary 311.

CE is generally referred to as a zone electrophoretic technique. There are many other electrophoretic techniques, however. cIEF is another electrophoretic technique, for example.

In cIEF, as in CE, a sample is injected at one end of capillary 311. After the sample is injected in cIEF, however, an acid is added to reservoir 315 and a base is added to reservoir 316. As a result, a pH gradient is established across capillary 311 in cIEF in addition to the voltage gradient provided by high voltage power supply 313.

Sample near the acidic side of capillary 311 is positively charged, for example. Sample near the basic side of capillary 311 is negatively charged. Due to the voltage gradient, the two types of sample migrate. The negative and positive charges move in opposite directions until they reach that point where the sample is neutrally charged because it has an equal number of positive and negative sides. At that point, the sample has a net zero charge. It does not move any further and stays put. Different compounds or proteins with different pH values are, therefore, focused into different bands in 330 in capillary 311, for example.

As a result, the cIEF experiment places compounds or proteins in bands 330 that correspond to different pH levels or pI values 340. This placing of compounds or proteins in fixed positions is performed in a focusing step, for example.

In order to detect bands 330, however, an additional detection step is performed. In the detection step, for example, a base is added to one end of capillary 311. At that point, the pH gradient breaks down and bands 330 start moving toward detector 320.

Different detectors may be used to analyze the sample after the electrophoretic separation has occurred. These detectors can include, but are not limited to, an ultraviolet (UV) detector, a laser-induced fluorescence (LIF) detector, or a mass spectrometer. A UV detector, for example, is used to measure the amount of UV light absorbed by the separated sample. An LIF detector, for example, is used to provide a high-sensitivity measurement of labeled molecular species.

In a system that combines CE or cIEF with electrospray ionization (ESI) and mass spectrometry (MS) or tandem mass spectrometry (MS/MS), the output of capillary 311 is input to an electrospray assembly (not shown). The electrospray ionization is accomplished by placing a high voltage potential at the outlet of the separation capillary with respect to the capillary inlet to the mass spectrometer. The separation capillary also requires a high voltage potential placed between its inlet and outlet. The separated portions of the sample are dispersed by the electrospray into a fine aerosol as they exit capillary 311. The droplets of the aerosol are then observed by a mass spectrometer or tandem mass spectrometer.

Compared to the early developmental instruments, fully automated CE or cIEF devices offer computer control of all operations, pressure and electrokinetic injection, an autosampler and fraction collector, automated methods development, precise temperature control, and an advanced heat dissipation system. Automation is critical to CE since repeatable operation is required for precise quantitative analysis.

### Background on Mass Spectrometry Techniques

Mass spectrometers are often coupled with chromatography or other separation systems, such as a cIEF device, in order to identify and characterize compounds of interest from a sample. In such a coupled system, the eluting solvent is ionized and a series of mass spectra are obtained from the eluting solvent at specified time intervals called retention times. These retention times range from, for example, 1 second to 100 minutes or greater. The series of mass spectra form a chromatogram, or extracted ion chromatogram (XIC).

Peaks found in the XIC are used to identify or characterize a known peptide or compound in the sample. More particularly, the retention times of peaks and/or the area of peaks are used to identify or characterize (quantify) a known peptide or compound in the sample.

In traditional separation coupled mass spectrometry systems, a fragment or product ion of a known compound is selected for analysis. A tandem mass spectrometry or mass spectrometry/mass spectrometry (MS/MS) scan is then performed at each interval of the separation for a mass range that includes the product ion. The intensity of the product ion found in each MS/MS scan is collected over time and analyzed as a collection of spectra, or an XIC, for example.

In general, tandem mass spectrometry, or MS/MS, is a well-known technique for analyzing compounds. Tandem mass spectrometry involves ionization of one or more compounds from a sample, selection of one or more precursor ions of the one or more compounds, fragmentation of the one or more precursor ions into fragment or product ions, and mass analysis of the product ions.

Tandem mass spectrometry can provide both qualitative and quantitative information. The product ion spectrum can be used to identify a molecule of interest. The intensity of one or more product ions can be used to quantitate the amount of the compound present in a sample.

A large number of different types of experimental methods or workflows can be performed using a tandem mass spectrometer. Three broad categories of these workflows are targeted acquisition, information dependent acquisition (IDA) or data-dependent acquisition (DDA), and data-independent acquisition (DIA).

In a targeted acquisition method, one or more transitions of a precursor ion to a product ion are predefined for a compound of interest. As a sample is being introduced into the tandem mass spectrometer, the one or more transitions are interrogated or monitored during each time period or cycle of a plurality of time periods or cycles. In other words, the mass spectrometer selects and fragments the precursor ion of each transition and performs a targeted mass analysis only for the product ion of the transition. As a result, an intensity (a product ion intensity) is produced for each transition. Targeted acquisition methods include, but are not limited to, multiple reaction monitoring (MRM) and selected reaction monitoring (SRM).

In an IDA method, a user can specify criteria for performing an untargeted mass analysis of product ions, while a sample is being introduced into the tandem mass spectrometer. For example, in an IDA method, a precursor ion or mass spectrometry (MS) survey scan is performed to generate a precursor ion peak list. The user can select criteria to filter the peak list for a subset of the precursor ions on the peak list. MS/MS is then performed on each precursor ion of the subset of precursor ions. A product ion spectrum is produced for each precursor ion. MS/MS is repeatedly performed on the precursor ions of the subset of precursor ions as the sample is being introduced into the tandem mass spectrometer.

In proteomics and many other sample types, however, the complexity and dynamic range of compounds are very large. This poses challenges for traditional targeted and IDA methods, requiring very high-speed MS/MS acquisition to deeply interrogate the sample in order to both identify and quantify a broad range of analytes.

As a result, DIA methods, the third broad category of tandem mass spectrometry, were developed. These DIA methods have been used to increase the reproducibility and comprehensiveness of data collection from complex samples. DIA methods can also be called non-specific fragmentation methods. In a traditional DIA method, the actions of the tandem mass spectrometer are not varied among MS/NIS scans based on data acquired in a previous precursor or product ion scan. Instead, a precursor ion mass range is selected. A precursor ion mass selection window is then stepped across the precursor ion mass range. All precursor ions in the precursor ion mass selection window are fragmented and all of the product ions of all of the precursor ions in the precursor ion mass selection window are mass analyzed.

The precursor ion mass selection window used to scan the mass range can be very narrow so that the likelihood of multiple precursors within the window is small. This type of DIA method is called, for example, MS/MS^{ALL}. In an MS/MS^{ALL} method, a precursor ion mass selection window of about 1 amu is scanned or stepped across an entire mass range. A product ion spectrum is produced for each 1 amu precursor mass window. The time it takes to analyze or scan the entire mass range once is referred to as one scan cycle. Scanning a narrow precursor ion mass selection window across a wide precursor ion mass range during each cycle, however, is not practical for some instruments and experiments.

As a result, a larger precursor ion mass selection window, or selection window with a greater width, is stepped across the entire precursor mass range. This type of DIA method is called, for example, SWATH acquisition. In a SWATH acquisition, the precursor ion mass selection window stepped across the precursor mass range in each cycle may have a width of 5-25 amu, or even larger. Like the MS/MS^{ALL} method, all the precursor ions in each precursor ion mass selection window are fragmented, and all of the product ions of all of the precursor ions in each mass selection window are mass analyzed.

### SUMMARY

A system, method, and computer program product are disclosed for separating intensity peaks from data collected in a separation device coupled mass spectrometry experiment, in accordance with various embodiments. The system includes a separation device, an ion source, a mass spectrometer, and a processor.

The separation device separates one or more compounds from a sample over a time period. The ion source ionizes the separated one or more compounds received from the separation device, producing an ion beam of one or more precursor ions.

The mass spectrometer receives the ion beam from the ion source and mass analyzes a mass range of the ion beam at each time of a plurality of times of the time period. A 3-D array of intensity measurements measured as a function of m/z and time for the time period is produced.

The processor receives the 3-D array from the mass spectrometer. The processor converts the 3-D array to an n×m intensity matrix D. The n rows of the D matrix correspond to measured m/z values or values of a mass-related parameter calculated from the measured m/z values and the m columns correspond to the plurality of times or values of a time-related parameter calculated from the plurality of times.

The processor applies NMF to the D matrix to solve for an intensity matrix M and an intensity matrix A of the equation D=MA. The NMF is applied to produce in a row (i) of the A matrix intensity values for a peak (i) in terms of the plurality of times or the values of the time-related parameter that separates the peak (i) from the 3-D array. The NMF is also applied to produce in column (i) of the M matrix intensity values in terms of the measured m/z values or the values of the mass-related parameter corresponding to the peak (i).

These and other features of the applicant's teachings are set forth herein.

### BRIEF DESCRIPTION OF THE DRAWINGS AND APPENDICES

The skilled artisan will understand that the drawings, described below, are for illustration purposes only. The drawings are not intended to limit the scope of the present teachings in any way.

FIG. 1 is a block diagram that illustrates a computer system, upon which embodiments of the present teachings may be implemented.

FIG. 2 is an exemplary three-dimensional (3-D) plot from a single cIEF-MS measurement showing measured intensities as a function of measured m/z values and measured time values, upon which embodiments of the present teachings may be implemented.

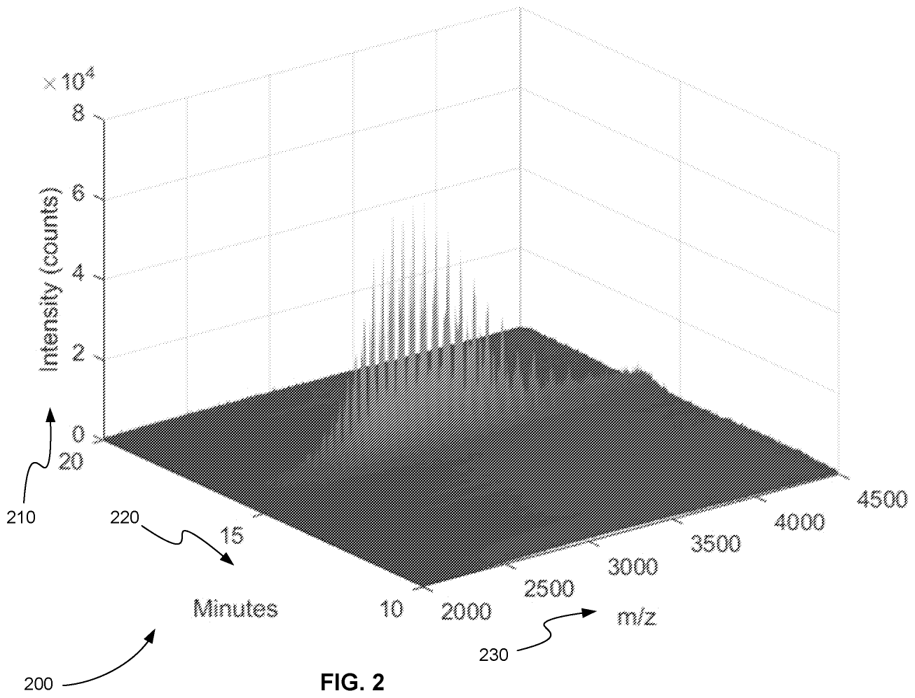

FIG. 3 is an exemplary schematic diagram of a capillary electrophoresis (CE) system, upon which embodiments of the present teachings may be implemented.

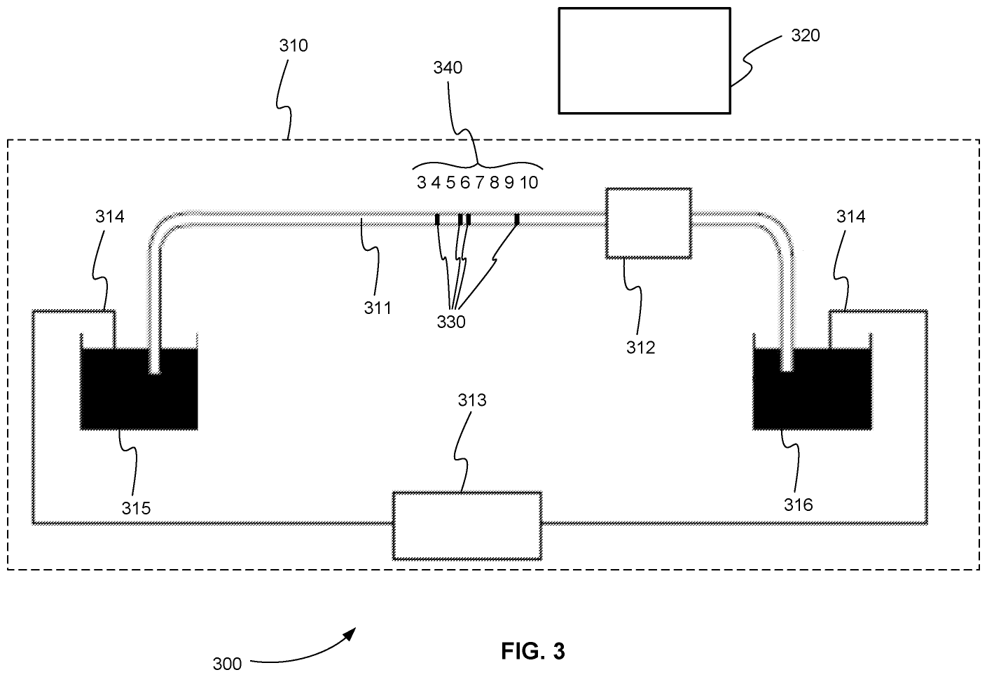

FIG. 4 is an exemplary diagram of a D matrix expressed as a matrix multiplication of an M matrix and an A matrix, in accordance with various embodiments.

FIG. 5 is an exemplary plot showing a reconstructed single electropherogram with peaks for all isoforms superimposed, in accordance with various embodiments.

FIG. 6 is a schematic diagram of a system for separating intensity peaks from data collected in a separation device coupled mass spectrometry experiment, in accordance with various embodiments.

FIG. 7 is a flowchart showing a method for separating intensity peaks from data collected in a separation device coupled mass spectrometry experiment, in accordance with various embodiments.

FIG. 8 is a schematic diagram of a system that includes one or more distinct software modules that perform a method for separating intensity peaks from data collected in a separation device coupled mass spectrometry experiment, in accordance with various embodiments.

Appendix 1 is an exemplary technical report paper describing a method for extracting cIEF-MS profiles from m/z and time arrays, in accordance with various embodiments.

Appendix 2 is an exemplary presentation describing a method for extracting cIEF-MS profiles from m/z and time arrays, in accordance with various embodiments.

Before one or more embodiments of the present teachings are described in detail, one skilled in the art will appreciate that the present teachings are not limited in their application to the details of construction, the arrangements of components, and the arrangement of steps set forth in the following detailed description or illustrated in the drawings. Also, it is to be understood that the phraseology and terminology used herein is for the purpose of description and should not be regarded as limiting.

### DESCRIPTION OF VARIOUS EMBODIMENTS

### Computer-Implemented System

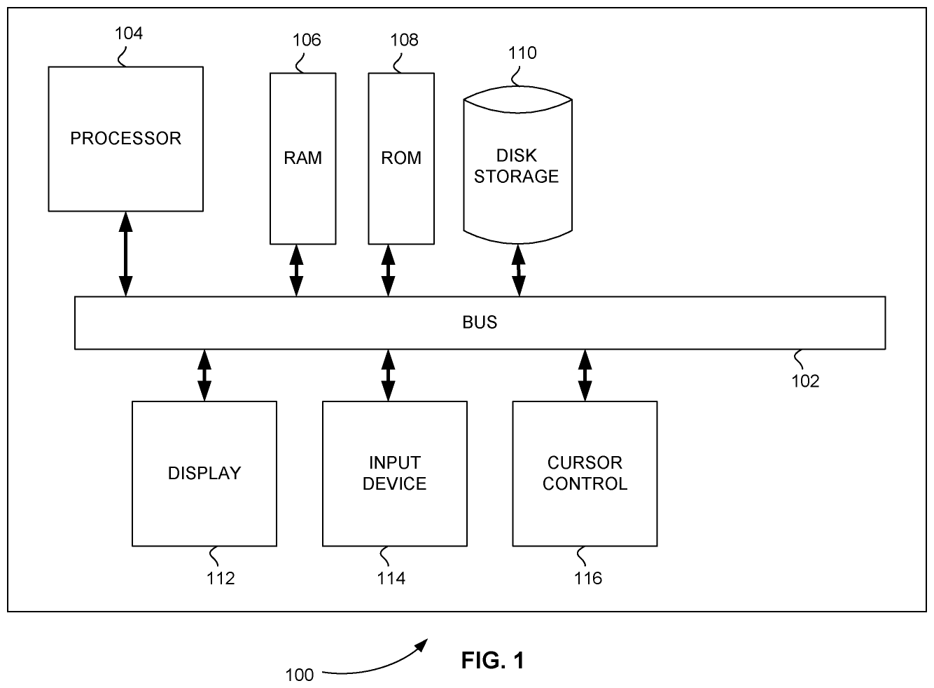

FIG. 1 is a block diagram that illustrates a computer system 100, upon which embodiments of the present teachings may be implemented. Computer system 100 includes a bus 102 or other communication mechanism for communicating information, and a processor 104 coupled with bus 102 for processing information. Computer system 100 also includes a memory 106, which can be a random-access memory (RAM) or other dynamic storage device, coupled to bus 102 for storing instructions to be executed by processor 104. Memory 106 also may be used for storing temporary variables or other intermediate information during execution of instructions to be executed by processor 104. Computer system 100 further includes a read-only memory (ROM) 108 or other static storage device coupled to bus 102 for storing static information and instructions for processor 104. A storage device 110, such as a magnetic disk or optical disk, is provided and coupled to bus 102 for storing information and instructions.

Computer system 100 may be coupled via bus 102 to a display 112, such as a cathode ray tube (CRT) or liquid crystal display (LCD), for displaying information to a computer user. An input device 114, including alphanumeric and other keys, is coupled to bus 102 for communicating information and command selections to processor 104. Another type of user input device is cursor control 116, such as a mouse, a trackball or cursor direction keys for communicating direction information and command selections to processor 104 and for controlling cursor movement on display 112. This input device typically has two degrees of freedom in two axes, a first axis (i.e., x) and a second axis (i.e., y), that allows the device to specify positions in a plane.

A computer system 100 can perform the present teachings. Consistent with certain implementations of the present teachings, results are provided by computer system 100 in response to processor 104 executing one or more sequences of one or more instructions contained in memory 106. Such instructions may be read into memory 106 from another computer-readable medium, such as storage device 110. Execution of the sequences of instructions contained in memory 106 causes processor 104 to perform the process described herein. Alternatively, hard-wired circuitry may be used in place of or in combination with software instructions to implement the present teachings. Thus, implementations of the present teachings are not limited to any specific combination of hardware circuitry and software.

In various embodiments, computer system 100 can be connected to one or more other computer systems, like computer system 100, across a network to form a networked system. The network can include a private network or a public network such as the Internet. In the networked system, one or more computer systems can store and serve the data to other computer systems. The one or more computer systems that store and serve the data can be referred to as servers or the cloud, in a cloud computing scenario. The one or more computer systems can include one or more web servers, for example. The other computer systems that send and receive data to and from the servers or the cloud can be referred to as client or cloud devices, for example.

The term “computer-readable medium” as used herein refers to any media that participates in providing instructions to processor 104 for execution. Such a medium may take many forms, including but not limited to, non-volatile media, volatile media, and transmission media. Non-volatile media includes, for example, optical or magnetic disks, such as storage device 110. Volatile media includes dynamic memory, such as memory 106. Transmission media includes coaxial cables, copper wire, and fiber optics, including the wires that comprise bus 102.

Common forms of computer-readable media or computer program products include, for example, a floppy disk, a flexible disk, hard disk, magnetic tape, or any other magnetic medium, a CD-ROM, digital video disc (DVD), a Blu-ray Disc, any other optical medium, a thumb drive, a memory card, a RAM, PROM, and EPROM, a FLASH-EPROM, any other memory chip or cartridge, or any other tangible medium from which a computer can read.

Various forms of computer-readable media may be involved in carrying one or more sequences of one or more instructions to processor 104 for execution. For example, the instructions may initially be carried on the magnetic disk of a remote computer. The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector coupled to bus 102 can receive the data carried in the infra-red signal and place the data on bus 102. Bus 102 carries the data to memory 106, from which processor 104 retrieves and executes the instructions. The instructions received by memory 106 may optionally be stored on storage device 110 either before or after execution by processor 104.

In accordance with various embodiments, instructions configured to be executed by a processor to perform a method are stored on a computer-readable medium. The computer-readable medium can be a device that stores digital information. For example, a computer-readable medium includes a compact disc read-only memory (CD-ROM) as is known in the art for storing software. The computer-readable medium is accessed by a processor suitable for executing instructions configured to be executed.

The following descriptions of various implementations of the present teachings have been presented for purposes of illustration and description. It is not exhaustive and does not limit the present teachings to the precise form disclosed. Modifications and variations are possible in light of the above teachings or may be acquired from practicing of the present teachings. Additionally, the described implementation includes software but the present teachings may be implemented as a combination of hardware and software or in hardware alone. The present teachings may be implemented with both object-oriented and non-object-oriented programming systems.

### Separating Peaks

Embodiments of systems and methods for separating intensity peaks from data collected in a separation device coupled mass spectrometry experiment, which includes the accompanying appendices. In this detailed description, for purposes of explanation, numerous specific details are set forth to provide a thorough understanding of embodiments of the present invention. One skilled in the art will appreciate, however, that embodiments of the present invention may be practiced without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of embodiments of the present invention.

Appendix 1 is an exemplary technical report paper describing a method for extracting cIEF-MS profiles from m/z and time arrays, in accordance with various embodiments.

Appendix 2 is an exemplary presentation describing a method for extracting cIEF-MS profiles from m/z and time arrays, in accordance with various embodiments.

As described above, capillary isoelectric focusing (cIEF) followed by mass spectrometry (cIEF-MS) is a hyphenated technique used to first separate a mixture of monoclonal antibodies or other proteins on the basis of their isoelectric points (pI) into isoforms. A single cIEF-MS measurement produces a large amount of three-dimensional (3-D) data, where the three dimensions are m/z, time, and intensity.

One problem in cIEF-MS data analysis is that this large amount of 3-D data needs to be reduced to a 2-D plot of intensity versus time or pI values. Another problem is that a parent mass value needs to be assigned to each important signal, or peak, in the 2-D plot.

Both of these problems can be overcome by conventional methods, if at every point along the time axis there is only a single protein isoform present at a unique pI value, or if every column of m/z data uniformly represents the ionic forms of a single unique parent mass. However, the separation of isoforms by cIEF is not perfect because any intensity value measured has the potential to originate from two or more isoforms having the same parent mass, two or more isoforms having unique parent masses, or two parent masses having pI values so similar that they are not well resolved.

In other words, reducing 3-D data collected from a separation device coupled MS or MS/MS experiment to 2-D data is conventionally confounded by peaks in the 3-D data that are convolved with respect to mass or time. As a result, systems and methods are needed to automatically reduce the 3-D to 2-D data even if peaks in the 3-D data are convolved with respect to mass or time.

In various embodiments, nonnegative matrix factorization (NMF) is applied to 3-D separation device coupled MS or MS/MS data to automatically reduce the 3-D to 2-D data even if peaks in the 3-D data are convolved with respect to mass or time. In the case of cIEF-MS, NMF can be applied to a matrix D, where the n rows of D correspond to m/z and the m columns of D correspond to time. Each element of D is an intensity. One skilled in the art can appreciate that the assignment of rows and columns are interchangeable. In other words, the n rows of D can alternatively be assigned time and the m columns of D can be assigned to m/z. Whichever way the rows and columns are assigned to the matrix D, the following steps need to follow that particular assignment.

In a preferred embodiment, the n rows of D correspond to parent mass and the m columns of D correspond to pI value. Again, the assignment of rows and columns is interchangeable. In this embodiment, measured m/z intensity data is first converted to parent mass at each point in time. This is done, for example, by convolving the intensity array along the m/z dimension using a binary kernel function that gives maximum response at a particular parent mass value. The m/z axis is replaced by the convolution of the intensity values with a set of kernel functions that span the expected parent mass range, which is 140 kD to 150 kD for monoclonal antibodies, for example.

This operation may be implemented by a single matrix multiplication of the intensity array with a kernel array. The results are approximate and, when more than one parent mass is represented in a single m/z scan, the convolution result will be a mixture of parent masses and not the desired single mass.

The time axis of the convolved intensity array is next mapped to pI. For example, the known m/z values of small peptide “pI markers” are used to determine the migration times of these markers, which are included in every sample for this purpose. Coincidence of two known m/z peaks is used to increase the discrimination of p1 markers from other components in the sample. Various regression methods then allow the derivation of a relationship between migration time and p1. Most commonly, linear regression is used but linear interpolation, spline fitting, and other methods are possible. At this point, the original intensity array has been converted to an array of “abundances” organized by approximate parent mass on one axis, and pI on the other. This is the matrix D.

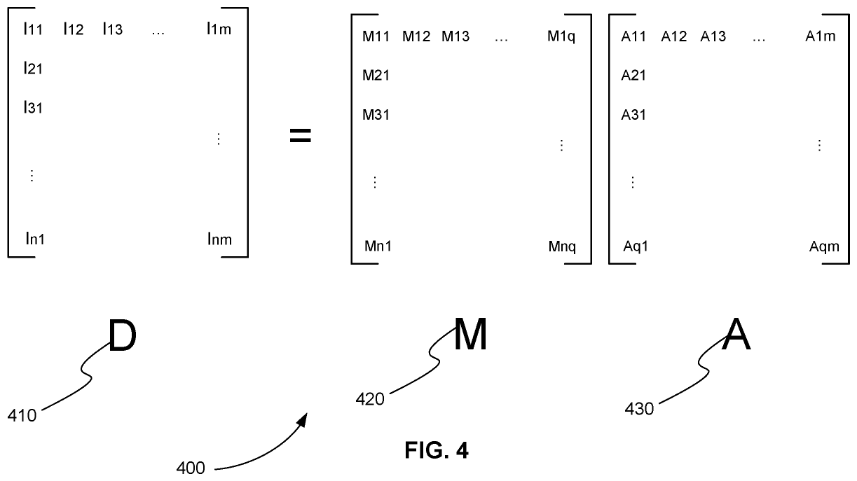

FIG. 4 is an exemplary diagram 400 of a D matrix expressed as a matrix multiplication of an M matrix and an A matrix, in accordance with various embodiments. NMF is now applied to D matrix 410. NMF makes use of the fact that real intensity values cannot be negative, nor can masses or relative abundances of a protein in the sample ever be negative. Furthermore, D matrix 410 is expressible as the matrix product D=MA, where matrix M 420 is a matrix of parent mass “spectra” and A matrix 430 is a matrix of isoform abundances. D matrix 410 has a number of rows, n, equal to the number of parent masses in the kernel function, and a number of columns, m, equal to the number of p1 values at which measurements were obtained. Matrices M 420 and A 430 need to have entirely nonnegative elements, and the number of components (columns in M 420 and rows in A 430) need to be kept to the smallest rank consistent with the actual number of distinct isoforms in the sample. Given D 410, the problem is to calculate optimal (or at least, reasonable) matrices M 420 and A 430, minimizing rank and enforcing nonnegativity.

Note that both M and A can have multiple columns and rows. If D has “n” rows and “m” columns, then M will have “n” rows and “q” columns, while A will have “q” rows and “m” columns. Each of the “q” columns of the matrix M (named because it contains mass, or m/z, data) is a vector that is representative of the m/z scan data (or mass spectrum) of the “qth” component. The value “n” is the integer number of m/z or mass channels scanned, for example. These column vectors are usually unit vectors, and their magnitude reflects only the relative counts or intensities in each channel, not the abundance of the component “q”. Each of the “q” rows of the matrix A (named because it contains information about the abundance of component “q”) is a vector that is representative of the intensity or counts associated with component “q” as a function of time, pI, etc. as disclosed, for example. Each of the “q” rows of A contains “m” values, which is the number of time points or pI values supplied in the matrix D, for example.

Advantageously, NMF objectively resolves the intensity data into the simplest component composition along the mass and pI axes simultaneously. At the conclusion of NMF, row (i) of A matrix 430 gives the unique isoform profile of isoform (i), and column (i) of M matrix 420 gives its parent mass reconstruction.

The results may be presented to the user as discrete profiles and reconstructed mass for each isoform (i) in this way. However, scientists are not used to seeing components of the profiles broken out in this way, since most cIEF methods provide a single “abundance” channel (usually an electropherogram) with peaks for all isoforms superimposed. To accommodate this convention, the column-wise sum of A matrix 430 may be presented as a single overall profile, with parent masses taken from the maxima of matrix M 420 and assigned correspondingly by component (i).

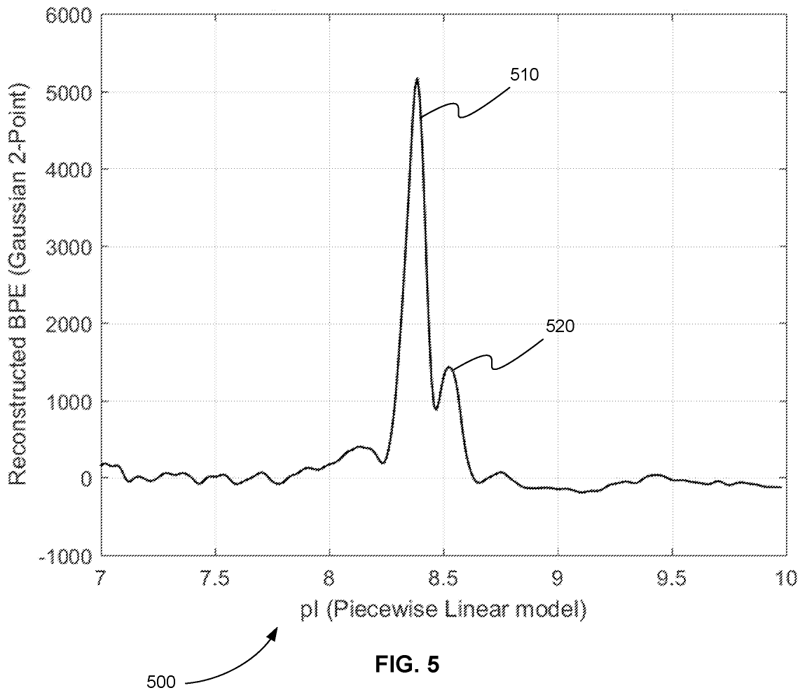

FIG. 5 is an exemplary plot 500 showing a reconstructed single electropherogram with peaks for all isoforms superimposed, in accordance with various embodiments. In plot 500, peaks 510 and 520 for different isoforms are shown.

In U.S. patent application Ser. No. 16/320,111 (hereinafter the “'111 Application”) a form of NMF referred to as nonnegative least squares (NNLS) was previously applied to a mass spectrometry method. In the '111 Application, NNLS was applied in a type of SWATH experiment referred to as scanning SWATH. In scanning SWATH, a large precursor ion selection window is scanned across a mass range of interest, producing a large number of overlapping precursor ion selection windows across the mass range. A product ion measurement is made for each overlapping precursor ion selection window and each product ion measurement is related to the location of its overlapping precursor ion selection window, producing a matrix relating product ion measurements to precursor ion location. NNLS was applied to the matrix to solve for a precursor ion column matrix. The '111 Application does not described or suggest applying NNLS to a matrix including separation device information and mass spectrometry information or separating peaks.

### System for Separating Peaks

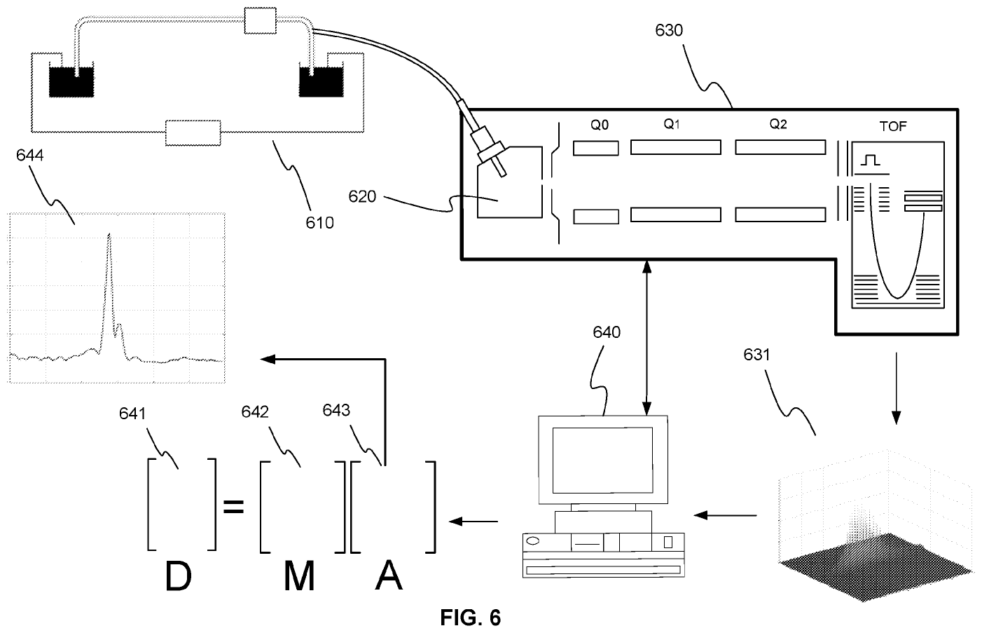

FIG. 6 is a schematic diagram 600 of a system for separating intensity peaks from data collected in a separation device coupled mass spectrometry experiment, in accordance with various embodiments. The system of FIG. 6 includes separation device 610, ion source 620, mass spectrometer 630, and processor 640.

Separation device 610 separates one or more compounds from a sample over a time period. A described above, the one or more compounds can include, for example, proteins such as monoclonal antibodies.

Separation device 610 is shown in FIG. 6 as a CE or cIEF device. However, separation device 620 can separate one or more compounds over time using one of a variety of other techniques. These techniques can include, but are not limited to, ion mobility, gas chromatography (GC), liquid chromatography (LC), or flow injection analysis (FIA).

Ion source 620 ionizes the separated one or more compounds received from separation device 610, producing an ion beam of one or more precursor ions. Ion source 620 is shown in FIG. 6 as performing electrospray ionization (ESI) (e.g., nanospray) but can be any type of ion source. Ion source 620 is also shown in FIG. 6 as being part of mass spectrometer 630. In various alternative embodiments, ion source 620 can be a device that is separate from mass spectrometer 630.

Mass spectrometer 630 receives the ion beam from ion source 620 and mass analyzes a mass range of the ion beam at each time of a plurality of times of the time period. A 3-D array 631 of intensity measurements measured as a function of m/z and time for the time period is produced. Mass spectrometer 630 mass analyzes a mass range of the ion beam at each time of a plurality of times of the time period by performing a mass spectrometry (MS) method or a mass spectrometry/mass spectrometry (MS/MS) method, for example.

Mass spectrometer 630 is shown in FIG. 6 as a quadrupole time-of-flight (QTOF) device. However, mass spectrometer 630 can be any type of mass spectrometer including, but not limited to, a quadrupole device, an ion trap device, a linear ion trap device, an orbitrap device, or a Fourier transform mass analyzer device.

Processor 640 is in communication with mass spectrometer 630. Processor 640 receives 3-D array 631 from mass spectrometer 630. Processor 640 converts 3-D array 631 to an n×m intensity matrix D 641. The n rows of D matrix 641 correspond to measured m/z values or values of a mass-related parameter calculated from the measured m/z values and the m columns correspond to the plurality of times or values of a time-related parameter calculated from the plurality of times.

Processor 640 applies nonnegative matrix factorization (NMF) to D matrix 641 to solve for an intensity matrix M 642 and an intensity matrix A 643 of the equation D=MA. The NMF is applied to produce in a row (i) of A matrix 643 intensity values for a peak (i) in terms of the plurality of times or the values of the time-related parameter that separates the peak (i) from 3-D array 631. The NMF is also applied to produce in column (i) of M matrix 642 intensity values in terms of the measured m/z values or the values of the mass-related parameter corresponding to the peak (i).

In various embodiments, while solving for M matrix 642 and A matrix 643, processor 640 enforces nonnegativity of elements of both M matrix 642 and A matrix 643 and minimizes columns of M matrix 642 and rows of A matrix 643 to minimize rank.

In various embodiments, processor 640 further displays on a display device (not shown) a peak profile of the peak (i) using information from A matrix 643 and M matrix 642. For example, processor 640 displays on the display device a peak profile of the peak (i) from the row (i) and an m/z or value of the mass-related parameter with the highest intensity from the column (i) for peak (i).

In various embodiments, processor 640 further displays on the display device a peak profile 644 of the peak (i) by combining or summing more than one row of A matrix 643, as described above. Specifically, processor 640 further sums the row (i) and one or more other rows of A matrix 643 and displays on the display device peak profile 644 of the peak (i) from the sum. FIG. 5, for example, shows a peak profile created by summing rows of A matrix 643.

Returning to FIG. 6, the display device can be a display device of processor 640, for example. In various alternative embodiments, the display device can be the display device of another device or a separate device.

In various embodiments, processor 640 can be a separate device as shown in FIG. 6 or can be a processor or controller of one or more devices of mass spectrometer 630, for example. Processor 640 can be, but is not limited to, a controller, a computer, a microprocessor, the computer system of FIG. 1, or any device capable of sending and receiving control signals and processing data.

### Method for Separating Peaks

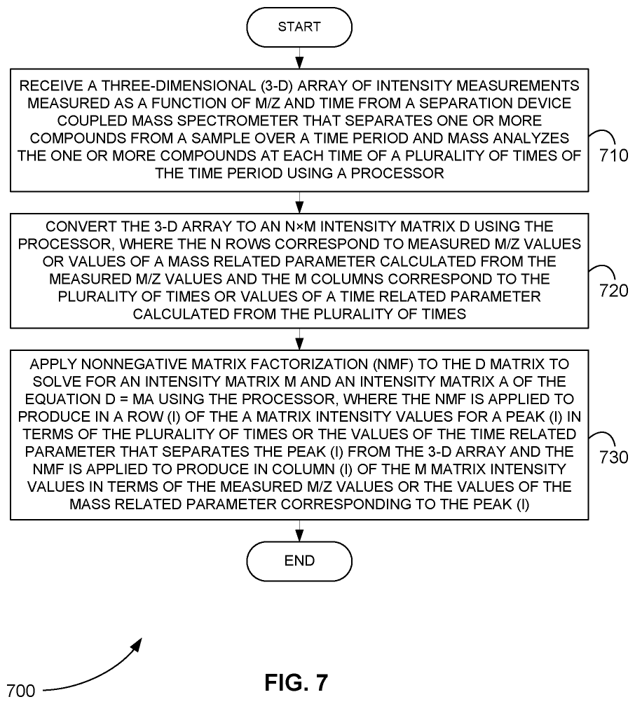

FIG. 7 is a flowchart showing a method 700 for separating intensity peaks from data collected in a separation device coupled mass spectrometry experiment, in accordance with various embodiments.

In step 710 of method 700, a three-dimensional (3-D) array of intensity measurements measured as a function of m/z and time is received from a separation device coupled mass spectrometer using a processor. The separation device coupled mass spectrometer separates one or more compounds from a sample over a time period and mass analyzes the one or more compounds at each time of a plurality of times of the time period.

In step 720, the 3-D array is converted to an n×m intensity matrix D using the processor. The n rows correspond to measured m/z values or values of a mass-related parameter calculated from the measured m/z values. The m columns correspond to the plurality of times or values of a time-related parameter calculated from the plurality of times.

In step 730, NMF is applied to the D matrix to solve for an intensity matrix M and an intensity matrix A of the equation D=MA using the processor. The NMF is applied to produce in a row (i) of the A matrix intensity values for a peak (i) in terms of the plurality of times or the values of the time-related parameter that separates the peak (i) from the 3-D array. The NMF is also applied to produce in column (i) of the M matrix intensity values in terms of the measured m/z values or the values of the mass-related parameter corresponding to the peak (i).

### Computer Program Product for Separating Peaks

In various embodiments, computer program products include a tangible computer-readable storage medium whose contents include a program with instructions being executed on a processor so as to perform a method for separating intensity peaks from data collected in a separation device coupled mass spectrometry experiment. This method is performed by a system that includes one or more distinct software modules.

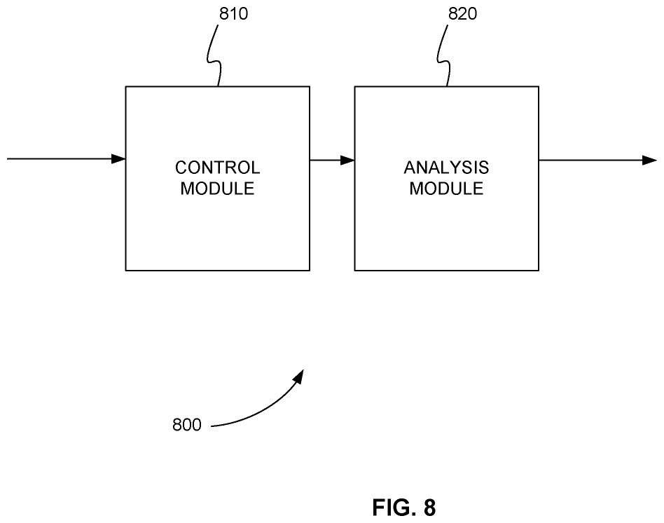

FIG. 8 is a schematic diagram of a system 800 that includes one or more distinct software modules that perform a method for separating intensity peaks from data collected in a separation device coupled mass spectrometry experiment, in accordance with various embodiments. System 800 includes a control module 810 and an analysis module 820.

Control module 810 instructs a separation device to separate one or more compounds from a sample over a time period. Control module 810 instructs an ion source to ionize the separated one or more compounds received from the separation device. An ion beam of one or more precursor ions is produced.

Control module 810 instructs a mass spectrometer to receive the ion beam from the ion source and mass analyze a mass range of the ion beam at each time of a plurality of times of the time period. A 3-D array of intensity measurements measured as a function of m/z and time for the time period are produced.

Analysis module 820 receives the 3-D array from the mass spectrometer. Analysis module 820 converts the 3-D array to an n×m intensity matrix D. The n rows correspond to measured m/z values or values of a mass-related parameter calculated from the measured m/z values. The m columns correspond to the plurality of times or values of a time-related parameter calculated from the plurality of times.

Analysis module 820 applies NMF to the D matrix to solve for an intensity matrix M and an intensity matrix A of the equation D=MA. The NMF is applied to produce in a row (i) of the A matrix intensity values for a peak (i) in terms of the plurality of times or the values of the time-related parameter that separates the peak (i) from the 3-D array. The NMF is also applied to produce in column (i) of the M matrix intensity values in terms of the measured m/z values or the values of the mass-related parameter corresponding to the peak (i).

While the present teachings are described in conjunction with various embodiments, it is not intended that the present teachings be limited to such embodiments. On the contrary, the present teachings encompass various alternatives, modifications, and equivalents, as will be appreciated by those of skill in the art.

Further, in describing various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments.

What is claimed is:

1. A system for separating intensity peaks from data collected in a separation device coupled mass spectrometry experiment, comprising:
a separation device that separates one or more compounds from a sample over a time period;
an ion source that ionizes the separated one or more compounds received from the separation device, producing an ion beam of one or more precursor ions;
a mass spectrometer that receives the ion beam from the ion source and mass analyzes a mass range of the ion beam at each time of a plurality of times of the time period, producing a three dimensional (3-D) array of intensity measurements measured as a function of m/z and time for the time period; and
a processor in communication with the mass spectrometer that
receives the 3-D array from the mass spectrometer,
converts the 3-D array to an n×m intensity matrix D, wherein the n rows correspond to measured m/z values or values of a mass-related parameter calculated from the measured m/z values and the m columns correspond to the plurality of times or values of a time-related parameter calculated from the plurality of times, and
applies nonnegative matrix factorization (NMF) to the D matrix to solve for an intensity matrix M and an intensity matrix A of the equation D=MA, wherein the NMF is applied to produce in a row (i) of the A matrix intensity values for a peak (i) in terms of the plurality of times or the values of the time-related parameter that separates the peak (i) from the 3-D array and the NMF is applied to produce in column (i) of the M matrix intensity values in terms of the measured m/z values or the values of the mass-related parameter corresponding to the peak (i).

2. The system of claim 1, wherein the separation device comprises a capillary isoelectric focusing (cIEF) device.

3. The system of claim 1, wherein the separation device comprises a capillary electrophoresis (CE) device, a liquid chromatography (LC) device, a gas chromatography (GC) device, an ion mobility device, or a flow injection analysis (FIA).

4. The system of claim 1, wherein the mass spectrometer mass analyzes a mass range of the ion beam at each time of a plurality of times of the time period by performing a mass spectrometry (MS) method or a mass spectrometry/mass spectrometry (MS/MS) method.

5. The system of claim 1, wherein, while solving for the M matrix and the A matrix, the processor enforces nonnegativity of elements of both the M matrix and the A matrix and minimizes columns of the M matrix and rows of the A matrix to minimize rank.

6. The system of claim 1, further comprising a display device that displays a peak profile of the peak (i) from the row (i) and displays an m/z or value of the mass-related parameter with the highest intensity from the column (i) for peak (i).

7. The system of claim 1, further comprising a display device, wherein the processor further sums the row(i) and one or more other rows of matrix A and displays on the display device a peak profile of the peak (i) from the sum.

8. The system of claim 1, wherein the n rows correspond to values of a mass-related parameter calculated from the measured m/z values and the m columns correspond to values of a time-related parameter calculated from the plurality of times and wherein the NMF is applied to produce in the row (i) intensity values in terms of the values of the time-related parameter for the peak (i) and the NMF is applied to produce in the column (i) intensity values in terms of the values of the mass-related parameter corresponding to the peak (i).

9. The system of claim 7, wherein the mass-related parameter comprises parent molecular mass and the processor calculates values for the parent molecular mass from the measured m/z values using a binary kernel function.

10. The system of claim 7, wherein the time-related parameter comprises isoelectric point (pI) and the processor calculates values for the pI using pI markers.

11. The system of claim 1, wherein the n rows correspond to measured m/z values and the m columns correspond to the plurality of times and wherein the NMF is applied to produce in the row (i) intensity values in terms of the plurality of times for the peak (i) and the NMF is applied to produce in the column (i) intensity values in terms of the measured m/z values corresponding to the peak (i).

12. The system of claim 10, wherein the processor further converts the row (i) intensity values in terms of the plurality of times for the peak (i) to intensity values in terms of isoelectric point (pI) using pI markers.

13. The system of claim 10, wherein the processor further converts the column (i) intensity values in terms of the measured m/z values corresponding to the peak (i) to intensity values in terms of parent molecular mass using a binary kernel function.

14. A method for separating intensity peaks from data collected in a separation device coupled mass spectrometry experiment, comprising:
receiving a three dimensional (3-D) array of intensity measurements measured as a function of m/z and time from a separation device coupled mass spectrometer that separates one or more compounds from a sample over a time period and mass analyzes the one or more compounds at each time of a plurality of times of the time period using a processor;
converting the 3-D array to an n×m intensity matrix D using the processor, wherein the n rows correspond to measured m/z values or values of a mass-related parameter calculated from the measured m/z values and the m columns correspond to the plurality of times or values of a time-related parameter calculated from the plurality of times, and
applying nonnegative matrix factorization (NMF) to the D matrix to solve for an intensity matrix M and an intensity matrix A of the equation D=MA using the processor, wherein the NMF is applied to produce in a row (i) of the A matrix intensity values for a peak (i) in terms of the plurality of times or the values of the time-related parameter that separates the peak (i) from the 3-D array and the NMF is applied to produce in column (i) of the M matrix intensity values in terms of the measured m/z values or the values of the mass-related parameter corresponding to the peak (i).

15. A computer program product, comprising a non-transitory and tangible computer-readable storage medium whose contents include a program with instructions being executed on a processor to perform a method for separating intensity peaks from data collected in a separation device coupled mass spectrometry experiment, the method comprising:
providing a system, wherein the system comprises one or more distinct software modules, and wherein the distinct software modules comprise a control module and an analysis module;
instructing a separation device to separate one or more compounds from a sample over a time period using the control module;
instructing an ion source to ionize the separated one or more compounds received from the separation device using the control module, producing an ion beam of one or more precursor ions;
instructing a mass spectrometer to receive the ion beam from the ion source and mass analyze a mass range of the ion beam at each time of a plurality of times of the time period using the control module, producing a three dimensional (3-D) array of intensity measurements measured as a function of mass-to-charge ratio (m/z) and time for the time period; and
receiving the 3-D array from the mass spectrometer using the analysis module;
converting the 3-D array to an n×m intensity matrix D using the analysis module, wherein the n rows correspond to measured m/z values or values of a mass-related parameter calculated from the measured m/z values and the m columns correspond to the plurality of times or values of a time-related parameter calculated from the plurality of times; and
applying nonnegative matrix factorization (NMF) to the D matrix to solve for an intensity matrix M and an intensity matrix A of the equation D=MA using the analysis module, wherein the NMF is applied to produce in a row (i) of the A matrix intensity values for a peak (i) in terms of the plurality of times or the values of the time-related parameter that separates the peak (i) from the 3-D array and the NMF is applied to produce in column (i) of the M matrix intensity values in terms of the measured m/z values or the values of the mass-related parameter corresponding to the peak (i).
